(19) United States
(12) Patent Application Publication
(10) Pub. No.: US 2010/0129414 A1
(43) Pub. Date: May 27, 2010

(54) Bioactive Agent Delivery Using Liposomes in Conjunction With Stent Deployment

(75) Inventors: Mark J. Dolan, Santa Rosa, CA (US); Dwayne S. Yamasaki, St. Augustine, FL (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa,, CA (US)

(21) Appl. No.: 12/276,995

(22) Filed: Nov. 24, 2008

(51) Int. Cl. A61F 2/04; A61K 9/127; A61K 39/395; A61K 31/7088; A61K 31/436; A61K 35/00

(52) U.S. Cl. 424/423; 424/450; 424/130.1; 514/291; 424/93.1; 514/44 R; 514/44 A

(57) ABSTRACT

Described herein are methods for treating aneurysms, vascular occlusions, and vascular lesions. The methods comprise the use of an implantable medical device which includes a bioactive agent substrate associated with its surface. Liposomes are used to encapsulate the bioactive agent and are delivered either systemically or locally to the bloodstream. A means for liberating the bioactive agents from the liposomes is used once an appropriate location is chosen and the liposomes have distributed themselves through the vasculature. Once liberated, the bioactive agent can be sequestered by the bioactive agent substrate associated with the implantable medical device, and slowly released to impart a therapeutic effect on the surrounding tissues.

### FIELD OF THE INVENTION

Described herein are methods for treating diseased blood vessels using a bioactive agent delivered in a liposome to an implantable medical device. The bioactive agent can be liberated from the liposome at the location of the implantable medical device and can be sequestered by a bioactive agent substrate associated with the implantable medical device.

### SUMMARY

Described herein are methods for treating aneurysms, vascular occlusions, and vascular lesions by administering bioactive agents in conjunction with the use of an implantable medical device. The methods generally comprise an implantable medical device with a coating thereon, where the coating comprises a bioactive agent substrate which has an affinity for at least one bioactive agent. The bioactive agent substrate is typically a protein or peptide such as an antibody or chemo-attractant agent. The implantable medical device is first implanted at a location in need of intervention. Then, liposomes containing one or more bioactive agent are administered to the bloodstream. The liposomes can be administered either locally or systemically depending on, for example, the toxicity of the bioactive agent. A location is then chosen that is adjacent to and upstream from the implanted medical device where a means for liberating the bioactive agents is used to release the bioactive agents from the liposomes. After the bioactive agents are liberated from the liposomes, they can be sequestered by the bioactive agent substrate associated with the implantable medical device and released to provide a therapeutic effect to the surrounding tissues.

In one embodiment herein, a method is described for treating a vessel in a patient in need thereof comprising the steps of: (a) providing an implantable medical device, wherein the implantable medical device comprises at least one bioactive agent substrate; (b) implanting the implantable medical device at a first location; (c) providing liposomes comprising at least one bioactive agent to the vasculature of the patient; (d) allowing a time sufficient for the liposomes to disperse through the vasculature of the patient; (e) using the first location of the implantable medical device to determine a second location adjacent to and upstream from the first location; and (f) directing energy to the second location of the liposomes present at the second location and liberating the at least one bioactive agent, whereby said at least one bioactive agent is sequestered by said bioactive agent substrate and released to provide a therapeutic effect to said vessel.

In one embodiment, the implantable medical device is selected from the group consisting of stents, sutures, catheters, micro-particles, probes, vascular grafts and combinations thereof.

In one embodiment, the at least one bioactive agent substrate is an antibody. In another embodiment, the antibody is specific for at least one of the bioactive agents. In one embodiment, the at least one bioactive agent substrate is a chemo-attractant. In another embodiment, the chemo-attractant compound is specific for at least one of the bioactive agents.

In one embodiment, the liposomes are echogenic. In another embodiment, the liposomes are delivered intravenously. In yet another embodiment, the liposomes are delivered locally.

In one embodiment, the at least one bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779), zotarolimus (ABT-578), cells and combinations thereof. In another embodiment, the cells are selected from the group consisting of embryonic cells, fetal cells, post-natal cells, adult stem cells, progenitor cells, cardiomyocytes, skeletal myocytes, skeletal myoblasts, mesenchymal stem cells, endothelial progenitor cells, hematological cells, immune cells, and combinations thereof.

In some embodiments, the method further comprises detecting the first location using fluoroscopy.

In one embodiment, the energy used to liberate the bioactive agents from the liposomes is selected from the group consisting of ultrasound, x-ray, radio frequency, infrared light, UV light, gamma rays, and electrical energy. In another embodiment, the energy is ultrasound between 250 kHz and 2000 kHz.

In one embodiment, the implantable medical device comprises a component to create turbulent flow at the upstream end of the implantable medical device. In another embodiment, the implantable medical device comprises at least one bioactive agent bound to the bioactive agent substrate prior to step (b).

In one embodiment of the present description, the method further comprises repeating steps (c) to (f) to recharge the bioactive agent substrate on the implantable medical device once the at least one bioactive agent has been at least partially depleted from the implantable medical device.

Further described herein is a method of providing bioactive agents to a vessel treated with an implantable medical device comprising the steps of: (a) providing a stent with at least one antibody associated with said stent; (b) implanting said stent in a vessel at a first location; (c) providing echogenic liposomes intravenously to the blood comprising at least one bioactive agent specific for said at least one antibody; (d) using said first location of said stent to determine a second location adjacent to and upstream from said first location; and (e) directing ultrasound between 250 kHz and 2000 kHz to said second location thereby bursting said liposomes present at said second location and releasing said at least one bioactive agent, whereby said at least one bioactive agent is sequestered by said at least one bioactive agent substrate and released to provide a therapeutic effect to said vessel.

### DEFINITIONS

Bioactive Agent: As used herein, “bioactive agent” can include any drug, pharmaceutical compound or molecule having a therapeutic effect in an animal and/or human. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms and cells.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, other rapamycin hydroxyesters may be used in combination with the terpolymers.

Biocompatible: As used herein “biocompatible” shall mean any material that does not cause injury or death to the animal or induce an adverse reaction in an animal when placed in intimate contact with the animal's tissues. Adverse reactions include, but are not limited to, inflammation, infection, fibrotic tissue formation, cell death and thrombosis.

Biodegradable: As used herein “biodegradable” refers to a polymeric composition that is biocompatible and subject to being broken down in vivo through the action of normal biochemical pathways. Bioresorbable and biodegradable may be used interchangeably, however they are not coextensive. Biodegradable polymers may or may not be reabsorbed into surrounding tissues. Bioresorbable polymers are biodegradable, and therefore, are capable of being cleaved into biocompatible byproducts through chemical- or enzyme-catalyzed hydrolysis.

Nonbiodegradable: As used herein, “nonbiodegradable” refers to a polymeric composition that is biocompatible and not subject to being broken down in vivo through the action of normal biochemical pathways.

Not Substantially Toxic: As used herein, “not substantially toxic” refers to systemic or localized toxicity, wherein the benefit to the recipient out-weighs the physiologically harmful effects of the treatment as determined by physicians and pharmacologists having ordinary skill in the art of toxicity.

Pharmaceutically Acceptable: As used herein, “pharmaceutically acceptable” refers to all compounds, prodrugs, derivatives and salts that are not substantially toxic at effective levels in vivo.

### DETAILED DESCRIPTION

Described herein are methods for treating vascular conditions utilizing administration of bioactive agents in conjunction with the use of an implantable medical device. Exemplary vascular conditions include, but are not limited to, aneurysms, vascular occlusions, and vascular lesions. The methods generally comprise an implantable medical device with a coating thereon, comprising a bioactive agent substrate which has an affinity for at least one bioactive agent. The first step of the method involves the implantation of the medical device. Then, liposomes for containing one or more bioactive agents are administered to the bloodstream. The toxicity of the bioactive agent determines whether the liposomes are administered locally or systemically. A location is then chosen that is adjacent to and upstream from the implanted medical device where a means for liberating the bioactive agents is used to release the bioactive agents from the liposomes and into the bloodstream. The location chosen for liberation of the bioactive agent from the liposomes can be highly dependent on the toxicity of the bioactive agent for the surrounding tissues. If, for example, the bioactive agent is highly toxic, the location of liberation may be very close to the implantable medical device. After the bioactive agents are liberated from the liposomes, they can flow to the implantable medical device in the direction of blood flow and be sequestered by the bioactive agent substrate associated with the implantable medical device wherein the bioactive agent is slowly released to provide a therapeutic effect to the surrounding tissues.

The methods described herein involve providing a suitable implantable medical device. Many implantable medical devices are known by those skilled in the art. Exemplary implantable medical devices include, but are not limited to, stents, catheters, sutures, micro-particles, probes and vascular grafts. The implantable medical devices can be used individually or in combination.

Additionally, implantable medical devices can be coated with one or more synthetic or natural substances by any means known in the art including, dipping, spraying, electrostatic deposition and brushing. The devices can be fully coated or partially coated. For example, a specific portion of a device can be coated with an appropriate polymer.

In one embodiment, natural and synthetic polymers can be used to coat the implantable medical devices. For example, the polymer chosen to coat the implantable medical devices can be biocompatible and may minimize irritation to the tissue. The biocompatible polymer can be either bioabsorbable or biostable depending on the desired rate of release and the desired degree of polymer stability.

Bioabsorbable polymers that can be used include, but are not limited to, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, polly sacharides or carbohydrates (i.e. starch, hyaluronic acids, dextran, heparin sulfate, chondoritin sulfate, heparin, alginate), proteins (i.e. polyamino alcohols, polyphosphazines, polyanhidrides), and collagen.

Conversely, biostable polymers with a relatively low chronic tissue response such as polyurethanes, silicones, and polyesters could be used. Other polymers could also be used if they can be dissolved and cured or polymerized on the medical device. Such polymers include, but are not limited to, polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The above polymers can be used to form a polymer matrix. The polymer matrix can be a hydrophobic matrix or can be a hydrophilic matrix. In one embodiment, the polymer matrix can comprise a combination of both hydrophobic and hydrophilic regions, wherein the regions can be utilized to create an appropriate balance of biocompatibility and bioactive agent encapsulation. For example, and not intended as a limitation, a hydrophobic region can be used to encapsulate a hydrophobic bioactive agent and a hydrophilic region can be used to provide the polymer with biocompatibility with an aqueous environment.

The implantable medical devices described herein comprise at least one bioactive agent substrate specific for at least one bioactive agent. The bioactive agent substrate can be associated with the surface of the implantable medical device. For example, but not intended as a limitation, the bioactive agent substrate can be coated on the surface of the device, it can be chemically bonded to the surface, it can be chemically bonded to a polymeric coating, and/or it can be intertwined into a polymer matrix associated with the device. The bioactive agent substrate can be specific for components other than bioactive agents including, but not limited to, small molecules, proteins, polymers, cells, microbes, antimicrobes, toxic substances, drugs, steroids, steroid like molecules, and the like. In one embodiment, the bioactive agent substrate is a protein such as a peptide, an antibody or a chemoattractant. In one embodiment, the antibody can be specific for a bioactive agent or a cell surface marker. In another embodiment, the antibody can be specific for a stem cell. In yet another embodiment, the chemo-attractant can be specific for a cell.

The antibodies described herein can comprise at least one type of antibody or fragment of an antibody. The antibody can be a monoclonal antibody, a polyclonal antibody, a chimeric antibody, or a humanized antibody. In one embodiment, the antibody or antibody fragment recognizes and binds a progenitor endothelial (endothelial cells, progenitor or stem cells with the capacity to become mature, functional endothelial cells) cell surface antigen and modulates the adherence of the cells onto the surface of the medical device. The antibody or antibody fragment can be covalently or non-covalently attached to the surface of the implantable medical device, or tethered covalently by a linker molecule to the outermost layer of the coating on the medical device. In this aspect, for example, the monoclonal antibodies can further comprise Fab or F(ab′)₂ fragments. The antibody fragment comprises any fragment size, which retains the characteristic of recognizing and binding the target antigen. In one embodiment, the antibody can be specific for a small molecule and/or bioactive agent. Methods of making antibodies specific for a small molecule and/or bioactive are well known in the art.

As used herein, the chemo-attractant includes any synthetic or natural molecule capable of attracting the desired cell type. In some embodiments, the chemo-attractant includes any synthetic or natural molecules capable of attracting an effective number of endothelial cells. The attractant generally has a degree of selectivity towards these cells. The chemo-attractant also includes any synthetic or natural molecules capable of binding to adhesion receptors differentially expressed on the endothelial cells. One such adhesion receptor is an integrin receptor. Some exemplary chemo-attractants include, but are not limited to, small integrin-binding molecules, arginine-glycine-aspartate (RGD) peptides or cyclic RGD peptides (cRGD), synthetic cyclic RGD (cRGD) mimetics, and small molecules binding to other adhesion receptors differentially expressed on the endothelial cells. In some embodiments, the chemo-attractant can specifically exclude a particular RGD peptide or cyclic RGD peptide.

In some embodiments, the chemo-attractants can be those molecules capable of binding to intercellular adhesion molecules (ICAM) or vascular cell adhesion molecules (VCAM), which are present in the endothelial cells. Such chemo-attractant can be, for example, receptors binding to ICAM or VCAM in the endothelial cells, which can include, but are not limited to, Decoy receptor 3 (DcR3), a tumor necrosis factor (TNF) that preferentially binds to ICAM and VCAM, β₂ integrin LFA-1, LFA-1Af (expressed on lymphocytes), or combinations thereof.

In one embodiment, the bioactive agent substrate can be attached to the surface of a medical device. In some of these embodiments, the surface can be a modified metallic surface or a polymeric surface if the medical device is formed of a polymer or is formed of a metal coated with a polymer. The bioactive agent substrate can be attached to the coating using a physical or chemical linkage.

Physical linkages can include hydrogen bonding, interpenetrating molecules or interpenetrating networks. Chemical linkages can use a linking agent or a direct bond between the surface and/or the coating material and the chemo-attractant and/or antibody.

In one embodiment, in addition to the therapeutic agent substrate, the implantable medical devices described herein can further comprise a bioactive agent for site specific delivery. The bioactive agents can be associated with the surface of the implantable medical device. In one embodiment, the polymers used to coat the implantable medical devices can comprise the one or more bioactive agents. The bioactive agents can be selected from anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) zotarolimus (ABT-578), somatic cells, stem cells and combinations thereof.

The stem cells used in the cell preparation include cells that proliferate and engraft into the myocardium of the patient and a physiologic carrier solution. The cells may be derived from a single individual or multiple individuals and may be of the same species or a different species than the recipient. In one embodiment, the cells are autologous.

Sources of cells suitable for use in the cell preparation can include, but are not limited to, embryonic, fetal, post-natal or adult stem or progenitor cells, cardiomyocytes, skeletal myocytes, skeletal myoblasts, mesenchymal stem cells, endothelial progenitor cells, hematological cells, immune cells, and combinations thereof. Sources of stem cells include, but are not limited to, bone marrow, blood, adipose tissue, gonads, skeletal or cardiac muscle, or any tissue containing stem cells. The cells may be obtained by any suitable method as would be known to persons of ordinary skill in the art.

The delivery of bioactive agents dispersed in polymer matrixes coated on the surface of implantable medical devices can be less than advantageous. Delivery of bioactive agents directly from polymer matrix coatings can be, in some embodiments, un-controllable, in that the bioactive agent is released over a predetermined time and once the bioactive agent is depleted, it is no longer available for further therapy. Additionally, some bioactive agents simply do not work well with implantable medical devices or the coatings applied to the surface of the implantable medical devices, or can cause irritation if exposed to the treatment site for long periods of time. For example, some bioactive agents dispersed in polymer matrixes coated on implantable medical devices have unpredictable release profiles and some may not survive the implantation process.

In contrast, the methods described herein allow bioactive agents to be delivered to the site of interest without being dispersed in a polymer matrix coated on the implantable medical device. Additionally, the methods described herein allow the delivery of bioactive agents to the implantable medical device without exposing the patient to systemic exposure to the agent as is the case with many implantable medical device therapies. Bioactive agents can be delivered in a liposome either locally or systemically and a means for liberating the bioactive agents can be provided to release the bioactive agent into the bloodstream.

The liposomes described herein have at least one bioactive agent associated with them and can be made of lipids such as, but not limited to, fatty acids, lysolipids, and phosphatidylcholine with both saturated and unsaturated lipids including dioleoylphosphatidylcholine; dimyristoylphosphatidylcholine; dipentadecanoylphosphatidylcholine, dilauroylphosphatidylcholine, dioleoylphosphatidylcholine, and dipalmitoylphosphatidylcholine; distearoylphosphatidylcholine; phosphatidylethanolamines such as dioleoylphosphatidylethanolamine; phosphatidylserine; phosphatidylglycerol; phosphatidylinositol and sphingolipids such as sphingomyelin; glycolipids such as ganglioside GM1 and GM2; glucolipids; sulfatides; glycosphingolipids; phosphatidic acid; palmitic acid; stearic acid; arachidonic acid; oleic acid; lipids bearing polymers such as polyethyleneglycol, chitin, hyaluronic acid and polyvinylpyrrolidone; lipids bearing sulfonated mono-, di-, oligo- or polysaccharides; cholesterol, cholesterol sulfate and cholesterol hemisuccinate; tocopherol hemisuccinate; lipids with ether and ester-linked fatty acids, polymerized lipids, diacetyl phosphate, stearylamine, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3β-yloxy)-1-thio-β-D-galactopyranoside, digalactosyldiglyceride, 6-(5-cholesten-3β-yloxy)hexyl-6-amino-6-deoxy-1-thio-α-D-galacto pyranoside, 6-(5-cholesten-3β-yloxy)hexyl-6-amino-6-deoxyl-1-thio-α-D-mannopyranoside, and 12-(((7′-diethylaminocoumarin-3-yl)carbonyl)methylamino)-octadecanoic acid; N-[1,2-(((7′-diethylaminocoumarin-3-yl)carbonyl)methyl-amino)octadecanoyl]-2-aminopalmitic acid; cholesteryl)4′-trimethyl-ammonio)butanoate; N-succinyldioleoylphosphatidylethanolamine; 1,2-dioleoyl-sn-glycerol;1,2-dipalmitoyl-sn-3-succinylglycerol; 1,3-dipalmitoyl-2-succinylglycerol;1-hexadecyl-2-palmitoylglycerophosphoet hanolamine and palmitoylhomocysteine, and/or combinations thereof. The liposomes can be formed as monolayers or bilayers and may or may not have a coating.

The liposomes can comprise a dose of at least one bioactive agent. In one embodiment, the dose can be from about 1 to about 1000 μg. In another embodiment, the dose can be from about 1 to about 500 μg, more preferably about 1 to about 250 μg, more preferably about 1 to about 100 μg. In another embodiment, the dose can be from about 500 to about 100 μg, more preferably about 750 to about 1000 μg, more preferably about 900 to about 1000 μg. In another embodiment, the dose can be from about 100 to about 900 μg, more preferably about 250 to about 750 μg, more preferably about 400 to about 600 μg. The dosages above can be per bioactive agent, if there is more than one, or can be a total dose of all bioactive agents.

The liposomes can have a gaseous interior or a fluid filled interior. If the interior is gaseous, the gas is preferably an inert gas such as, but not limited to, argon. Other gases which may be useful include air, oxygen, hydrogen, nitrogen, fluorocarbons, xenon, helium, and fluoropropanes. The bioactive agents associated with the liposomes can be encased within the liposomes, incorporated into the lipid layers making up the liposomes, attached to the inside of the liposomes, attached to the outside of the liposomes, or a combination thereof. Methods of preparing liposomes are commonly known in the art.

In one embodiment, a non-limiting example of a method for preparing liposomes comprises the steps of shaking an aqueous solution, comprising a lipid, in the presence of gas at a temperature below the gel to liquid crystalline phase transition temperature of the lipid to form gas-filled liposomes, and adding a bioactive agent. Such a method forms liposomes with a gaseous interior wherein the bioactive agent is contained.

In another embodiment, a method for preparing liposomes comprises the step of shaking an aqueous solution comprising a lipid and a bioactive agent in the presence of a gas at a temperature below the gel to liquid crystalline phase transition temperature of the lipid. In other embodiments, methods for preparing liposomes comprise the steps of shaking an aqueous solution, comprising a lipid and a bioactive agent, in the presence of a gaseous, and separating the resulting gas-filled liposomes for therapeutic use.

The liposomes described herein can be detectable. Detectability can be important if the distribution of the liposomes in the vasculature and/or tissues is important. Detection can be by a means such as, but not limited to, ultrasound, x-ray, radio frequency, infrared light, UV light, gamma rays, and electrical energy. The detection method should have energy sufficient to image the liposomes, but not serve as a means to liberate the bioactive agents from the liposomes. In one embodiment, the liposomes are echogenic and can be detected by ultrasound waves. In one embodiment, the ultrasound waves used to image the liposomes is between about 250 kHz and about 2000 kHz ranges. The frequency of ultrasound should be sufficient to image the liposomes, but not to rupture them.

The liposomes according to the methods described herein can be delivered intravenously or locally. If delivered to a patient intravenously, once the liposomes are injected, they begin circulating and dispersing into the vasculature. Since the liposomes can be re-circulated, they must sustain sufficient structural integrity allowing re-circulation without rupturing them. If the liposomes are delivered locally, the liposomes are delivered directly to the site of interest and ruptured accordingly thereby limiting possible systemic exposure. Methods of acute or local delivery can include, but are not limited to, injection and delivery via a catheter.

The methods described herein can further comprise detection of an implantable medical device's location, herein referred to as the first location. Since the intravenous delivery of the liposomes can be at a time after the actual implantation of the medical device, the location of the medical device may need to be re-established at the time of intravenous injection. A method of modality is used in detecting the location of the implanted medical device. In one exemplary embodiment, the method of modality is fluoroscopy. Other non-limiting methods can include magnetic resonance imaging (MRI), ultrasound, the use of radio frequency markers, and the use of Calypso transmitters.

Once the location of the implanted medical device has been established, the location can be used to establish a location adjacent to and upstream from the implanted medical device (herein referred to as the second location). The second location adjacent to the implanted medical device will provide a location for modification of the liposomes, which is discussed below. If the location of the implanted medical device is already known, then the second location can be chosen more easily. The location should be one that is upstream, opposite the direction of blood flow in the vessels, of the implanted medical device.

Once the second location adjacent to the implanted medical device is established, a means of liberating the bioactive agents from the liposomes is required. One means of liberation is energy directed at the second location. The energy should be sufficient to modify the liposomes. Modification can include, but is not limited to, rupturing, shaking off surface bound substances, exploding, and imploding.

In one embodiment, the location of the implanted medical device can be approximate and therefore, the energy can be applied to an approximate location as well. In one embodiment, the exact location need not be known at all. For example, the medical device can be a vascular stent and energy can be directed at the region of the chest or the region of the heart. Energy dispersed in the general area can be sufficient in some embodiments.

In one embodiment, the modification is by rupturing the liposomes, thereby releasing the bioactive agent contained therein into the vessel. The energy needed is applied to the second location which is adjacent to the implanted medical device. The location can be at a distance sufficient to allow the bioactive agent to completely disperse from the ruptured liposomes before reaching the implanted medical device (this will depend on the speed of blood flow in the vessel.)

In one embodiment, the energy can be provided from a local source at the location of implantation. For example, and not meant as a limitation, the energy can be provided by an energy source provided by a catheter.

In one embodiment, the frequency (energy) required to modify the liposomes is between about 1 kHz and about 10,000 kHz, more preferably between about 1 kHz and about 5,000 kHz, and most preferably between about 1 kHz and about 1,000 kHz. In another embodiment, the frequency (energy) required to rupture the liposomes is between about 1,000 kHz and about 10,000 kHz, more preferably between about 1,000 kHz and about 5,000 kHz, and most preferably between about 1,000 kHz and about 2,500 kHz.

Once the liposomes are modified and the bioactive agent is liberated, the bioactive agent can flow from the site of liposomal modification to the site of the implanted medical device by means of blood flow. The energy applied to the second location can be tailored to provide a specific amount of bioactive agent to the site of the implanted medical device. The concentration of liposomes can be determined, for example, by imaging as described supra. If the liposomes contain a known concentration of bioactive agent and a rupture energy ratio is known (a ratio of energy applied to percentage of bioactive agent released), a certain concentration of bioactive agent can be delivered to the site of implantation, simply by dialing in the correct energy (e.g. frequency) at a given location.

The intact liposomes, ruptured liposomes and released bioactive agents (the components) flow through the vasculature in the direction of blood flow. Therefore, the components will flow from the second location to the implantation site (first location). Once the components reach the first location, the bioactive agents can interact with the bioactive agent substrate associated with the implanted medical device surface and/or can interact with the surrounding tissues as needed.

In one embodiment, in addition to, or in conjunction with the implantable medical device is an apparatus or method of increasing turbulence to the blood flow or disrupting blood flow either upstream from the medical device or at the location of the medical device. The disruption of blood flow result from a component of the implantable medical device located at the upstream end of the implantable medical device. The component is used to create a turbulent blood flow as the blood passes through or around the device. It is understood that much of the vasculature, especially in regions without curves or bends, displays laminar flow there through. If the flow through the region wherein the implantable medical device is located is laminar, bioactive agents located at the center of the flow will have a very low probability of coming in contact with the medical device. The increase in blood flow turbulence will cause a mixing of the blood and allow the bioactive agent located at the center of the vessel to increase their probability of coming in contact with the implantable medical device, especially if it lines the walls of the vessel. As such, it would be advantageous for the blood flow to be disrupted to thoroughly mix the blood in the vessel to increase the probability of contact of the bioactive agent with the implantable medical device. In some embodiments, no disruption of blood flow or increase in turbulence is needed and natural blood flow is used according to the present disclosure.

Devices that can accomplish this task of disruption blood flow or in creasing turbulence in the vessel include balloons, such as balloon catheters; catheters with disruption devices such as dimples and protrusions; screw shaped devices; blood flow diversion devices such as levies; and impellers or propellers. Methods can also be used to accomplish this task of disruption blood flow or in creasing turbulence in the vessel such as physical activity of the patient, such as walking, jogging, exercise, and general movement, and applying energy such as ultrasound to the region.

In one embodiment, the implantable medical device can be coated with several different types of antibodies specific for different bioactive agents. Liposomes with the specific bioactive agents can be introduced by intravascular administration. The liposomes can be ruptured at a location adjacent to the implanted, coated medical device. The different bioactive agents can be attracted to antibodies specific for that bioactive agent. Strategic antibody coatings at specific locations on the implantable medical device can be an important aspect of the present technology.

In one embodiment, the process of delivering bioactive agents to an implantable medical device can be repeated after a bioactive agent has performed its function at the implantation site and has been exhausted leaving free bioactive agent substrate. For example, once the bioactive agent has been exhausted at the site of implantation, the process can be repeated to “recharge” the medical device with a new, “fresh” bioactive agent. This can allow for multiple treatments using a single implanted medical device. In one embodiment, wherein the implantable medical device is coated with several different bioactive agent substrates, bioactive agents can be delivered in different phases or at different times as needed. One skilled in the art can evaluate a patient's condition and formulate a delivery regiment of bioactive agent(s) that will provide the most value to a patient's condition.

It is understood by those skilled in the art that certain types of implantable medical devices are endothelialized once implanted and such devices once endothelialized may not be as susceptible to recharging procedures. For such devices, it is understood that recharging is appropriate as long as there are portions of the device not endothelialized and still retain a bioactive agent substrate thereon. If a device is not endothelialized, it can be recharged as long as there remains adequate bioactive agent substrate.

In one embodiment, the implantable medical device can be charged with a bioactive agent before the device is implanted, during implantation in a catheter or locally, immediately after implantation. If the device is charged with a bioactive agent before implantation, the bioactive agent is attached to the bioactive agent substrate before implantation (e.g. on site or packaged from the factory) and once implanted, can begin t controllably release bioactive agent. Once at lease a portion of bioactive agent has been exhausted from the implantable medical device, the bioactive agent can be recharged one or more times as described above.

The device, if it is intravascular device, can be charged with a bioactive agent within a catheter during the implantation procedure. The device including a bioactive agent substrate is submerged in a bioactive agent (e.g. solution containing a bioactive agent) within a catheter and the bioactive agent is sequestered by the bioactive agent substrate. Once the device has been coated, it is inserted into the vessel using the catheter. Once the device is implanted, it can be recharged one or more times as described above.

The device can also be charged locally just after implantation. The implantable medical device including a bioactive agent substrate is implanted. Bioactive agent is delivered locally to provide an initial charge of bioactive agent to the implantable medical device. Once the charge is completed, the implantable medical device provides a therapeutics effect and can be recharged one or more times as described above. In one embodiment, the bioactive agent can be delivered locally to the vessel by the catheter during the implantation procedure.

### EXAMPLE 1

### Metal Stent Cleaning Procedure

Stainless steel stents are placed in a glass beaker and covered with reagent grade or better hexane. The beaker containing the hexane-immersed stents is then placed into an ultrasonic water bath and treated for 15 minutes at a frequency of between approximately 25 to 50 KHz. Next the stents are removed from the hexane and the hexane was discarded. The stents are then immersed in reagent grade or better 2-propanol and vessel containing the stents and the 2-propanol was treated in an ultrasonic water bath as before. Following cleaning the stents with organic solvents, they are thoroughly washed with distilled water and thereafter immersed in 1.0 N sodium hydroxide solution and treated at in an ultrasonic water bath as before. Finally, the stents are removed from the sodium hydroxide, thoroughly rinsed in distilled water and then dried in a vacuum oven over night at 40° C. After cooling the dried stents to room temperature in a desiccated environment they are weighed and their weights are recorded.

### EXAMPLE 2

### Coating a Clean, Dried Stent Using a Polymer

In the following Example, ethanol is chosen as the solvent of choice. Other solvents within the knowledge of those skilled in the art are within the scope of the present description. Persons having ordinary skill in the art of polymer chemistry can easily pair the appropriate solvent system to the polymer and achieve optimum results with no more than routine experimentation.

Antibodies specific for rapamycin are acquired by methods commonly known in the art and added to a solvent and mixed until the compounds are dissolved. Then, polycaprolactone (PCL) is added to the antibody solution and mixed until the PCL dissolved forming an antibody/polymer solution.

The cleaned, dried stents are coated using either spraying techniques or dipped into the antibody/polymer solution. The stents are coated as necessary to achieve a final coating weight of between approximately 10 μg to 1 mg. Finally, the coated stents are dried in a vacuum oven at 50° C. overnight. The dried, coated stents are weighed and the weights recorded.

### EXAMPLE 3

### Generating Loaded Liposomes

Liposomes are prepared comprising the steps of shaking an aqueous solution, comprising phosphatidylcholine and rapamycin, in the presence of argon at a temperature of 10° C.

### EXAMPLE 4

### Administering Liposomes

Liposomes prepared according to Example 3 are administered intravenously to the patient. The liposomes are allowed to circulate the body for about 60 minutes. The liposomes are imaged and mapped using ultrasound. The location of the stent can also be identified using similar ultrasound techniques.

### EXAMPLE 5

### Liberating Bioactive Agents from Liposomes and Effected Delivery

Knowing the location of the implanted stent (first location), a second location adjacent to the stent in a direction against blood flow is chosen. Ultrasound of about 1000 kHz is directed at the location adjacent to the stent location. The ultrasound waves at a frequency of 1000 kHz are sufficient to liberate about 99% of liposomes passing through that section of vasculature.

Upon liberation, the rapamycin encased in the liposomes is released into the surrounding hemodynamic environment and flow in the blood flow's direction arriving at the location of the implanted stent. The antibodies coated on the stent attract the rapamycin and the rapamycin is retained on the stent coating. Once bound to the stent, the rapamycin can affect a response on the stented vessel.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A method of treating a vessel in a patient in need thereof comprising the steps of:
(a) providing an implantable medical device wherein said implantable medical device comprises at least one bioactive agent substrate;
(b) implanting said implantable medical device at a first location;
(c) providing liposomes comprising at least one bioactive agent capable of binding to said at least one bioactive agent substrate to the vasculature of said patient;
(d) allowing a time sufficient for said liposomes to disperse through the vasculature of said patient;
(e) using said first location of said implantable medical device to determine a second location adjacent to and upstream from said first location; and
(f) directing energy to said liposomes present at said second location, thereby liberating said at least one bioactive agent from the liposomes and into said vessel, whereby said at least one bioactive agent is sequestered by said at least one bioactive agent substrate on the implanted device, and subsequently released to provide a therapeutic effect to said vessel.

2. The method according to claim 1 wherein said implantable medical device is selected from the group consisting of stents, sutures, catheters, micro-particles, probes, vascular grafts and combinations thereof.

3. The method according to claim 1 wherein said at least one bioactive agent substrate is an antibody.

4. The method according to claim 3 wherein said antibody is specific for at least one of said bioactive agents.

5. The method according to claim 1 wherein said at least one bioactive agent substrate is a chemo-attractant.

6. The method according to claim 5 wherein said chemo-attractant compound is specific for at least one of said bioactive agents.

7. The method according to claim 1 wherein said liposomes are echogenic.

8. The method according to claim 7 wherein said liposomes are delivered intravenously.

9. The method according to claim 7 wherein said liposomes are delivered locally.

10. The method according to claim 1 wherein said at least one bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779), zotarolimus (ABT-578), and cells.

11. The method according to claim 10 wherein said cells are selected from the group consisting of embryonic cells, fetal cells, post-natal cells, adult stem cells, progenitor cells, cardiomyocytes, skeletal myocytes, skeletal myoblasts, mesenchymal stem cells, endothelial progenitor cells, hematological cells, immune cells, and combinations thereof.

12. The method according to claim 1 wherein said method further comprises detecting said first location using fluoroscopy.

13. The method according to claim 1 wherein said energy is selected from the group consisting of ultrasound, x-ray, radio frequency, infrared light, UV light, gamma rays, and electrical energy.

14. The method according to claim 12 wherein said energy is ultrasound.

15. The method according to claim 13 wherein said ultrasound is between 250 kHz and 2000 kHz.

16. The method according to claim 1 wherein said implantable medical device comprises a component to create turbulent flow at the upstream end of said implantable medical device.

17. The method according to claim 1 wherein said implantable medical device comprises at least one bioactive agent bound to said bioactive agent substrate prior to step (b).

18. The method according to claim 1 further comprising repeating steps (c) to (f) to recharge said bioactive agent substrate on said implantable medical device once said bioactive agent has been at least partially depleted from said implantable medical device.

19. A method of providing bioactive agents to a vessel treated with an implantable medical device comprising the steps of:
(a) providing a stent with at least one antibody associated with said stent;
(b) implanting said stent in a vessel at a first location;
(c) providing echogenic liposomes intravenously to the blood comprising at least one bioactive agent specific for said at least one antibody;
(d) using said first location of said stent to determine a second location adjacent to and upstream from said first location; and
(e) directing ultrasound between 250 kHz and 2000 kHz to said second location thereby bursting said liposomes present at said second location and releasing said at least one bioactive agent into said vessel, wherein said at least one bioactive agent is sequestered by said at least one antibody and released to provide a therapeutic effect to said vessel.
